(19) United States
(12) Patent Application Publication
(10) Pub. No.: US 2018/0049723 A1
(43) Pub. Date: Feb. 22, 2018

(54) SMARTPHONE ENABLED URINALYSIS DEVICE, SOFTWARE, AND TEST PLATFORM

(71) Applicant: Wellmetris, LLC, Keego Harbor, MI (US)

(72) Inventor: Dahl Andrew A., Bloomfield Hills, MI (US)

(21) Appl. No.: 15/560,989

(22) PCT Filed: Mar. 23, 2016
(86) PCT No.: PCT/US2016/023702

§ 371 Date: Sep. 22, 2017

Related U.S. Application Data
(60) Provisional application No. 62/136,764, filed on Mar. 23, 2015.

(51) Int. Cl. A61B 10/00; A61B 5/145; A61B 5/00; G01N 33/493; A61B 5/20; G01N 21/01; G01N 33/50

(52) U.S. Cl. CPC A61B 10/007; A61B 5/14507; A61B 5/6898; G01N 33/493; G01N 21/8483; A61B 5/20; G01N 21/01; G01N 33/50; A61B 5/14546

(57) ABSTRACT

A urinalysis device including a device body having a port for electronic communication with a handheld computing device and holding a test strip for insertion into urine and collecting a sample. A method of using the urinalysis device with a handheld computing device, by taking a urine sample, electronically coupling the urinalysis device to the handheld computing device through the port, obtaining raw data a test strip within the urinalysis device, sending the raw data to the handheld computing device, and further analyzing the raw data. An Application and method of use with a urinalysis device, including a power providing mechanism for recognizing and providing power to the urinalysis device, a data accepting mechanism for accepting raw data from the urinalysis device, a data correcting mechanism for converting and correcting raw data from the urinalysis device to results, and a reporting mechanism for populating a reporting form with results.

### BACKGROUND OF THE INVENTION

### 1. Technical Field

The present invention relates to devices for collecting urine and other fluids, and in particular to devices that couple to smartphones and other handheld computing devices.

### 2. Background Art

Urine and other body fluids are frequently collected for analysis, for purposes of medical diagnosis, and wellness monitoring. In many types of analysis, a sample of collected fluid is applied to a test strip. The fluid reacts with analytic reagents that are either present in the test strip, or introduced at a later point in time.

Handheld devices are being developed that can be placed directly in a urine stream to take a sample. For example, CN102520145 discloses a handheld urine detecting device, which includes a handheld pen body and a pen cap, wherein the pen cap is detachably connected and fixed at the head end or tail end of the handheld pen body; a urine absorbing block and a piece of urine detecting test paper are arranged in the handheld pen body; the urine absorbing block extends out of the head end of the handheld pen body; a supporting frame is arranged on a side surface of the handheld pen body; and the tail end of the handheld pen body is bent toward the side of the supporting frame. When a user uses the handheld device, the user can hold the bent tail end of the handheld pen body in the handheld device and pour the urine onto the urine absorbing block of the handheld pen body so as to take urine. In general with these devices, the test strip with the urine sample is placed in a separate reader connected to or containing its own computer for analysis.

It would be advantageous to further add portability and analysis functions to handheld urine devices especially with the prevalence of smartphones and other handheld computing devices.

### SUMMARY OF THE INVENTION

The present invention provides for a urinalysis device including a device body having a port for electronic communication with a handheld computing device and holding a test strip for insertion into urine and collecting a sample.

The present invention also provides for a method of using the urinalysis device with a handheld computing device, by taking a urine sample with the urinalysis device, electronically coupling the urinalysis device to the handheld computing device through the port, obtaining raw data a test strip within the urinalysis device, sending the raw data to the handheld computing device, and further analyzing the raw data.

The present invention provides for an Application for use with a urinalysis device on non-transitory computer readable media, including a power providing mechanism for recognizing and providing power to the urinalysis device, a data accepting mechanism for accepting raw data from the urinalysis device, a data correcting mechanism for converting and correcting raw data from the urinalysis device to results, and a reporting mechanism for populating a reporting form resident in the Application with results.

The present invention provides for a method of using a urinalysis device with an Application on non-transitory computer readable media, including the steps of recognizing and providing power to the urinalysis device, accepting raw data from the urinalysis device, converting/correcting raw data from the urinalysis device to results, and populating a reporting form resident in the Application with results.

### DESCRIPTION OF THE DRAWINGS

Other advantages of the present invention are readily appreciated as the same becomes better understood by reference to the following detailed description when considered in connection with the accompanying drawings wherein:

FIG. 1 is a top perspective view of an urinalysis device coupled to a smartphone;

FIG. 2 is a top view of an urinalysis device coupled to a smartphone;

FIG. 3 is a side view of an urinalysis device coupled to a smartphone;

FIG. 4 is a close-up view of a urinalysis device coupled to a smartphone;

FIG. 5 is front perspective view of an urinalysis device coupled to a smartphone;

FIG. 6 is a bottom perspective view of an urinalysis device coupled to a smartphone;

FIG. 7 is a top perspective view of an urinalysis device coupled to a smartphone;

FIG. 8 is a flowchart of the processes within an App of the present invention;

FIG. 9 is a photograph of a test strip withdrawn from the urinalysis device;

FIG. 10 is a photograph of a top view of the urinalysis device coupled to a smartphone; and

FIG. 11 is a schematic of the flow of information between the App and the urinalysis device.

### DETAILED DESCRIPTION OF THE INVENTION

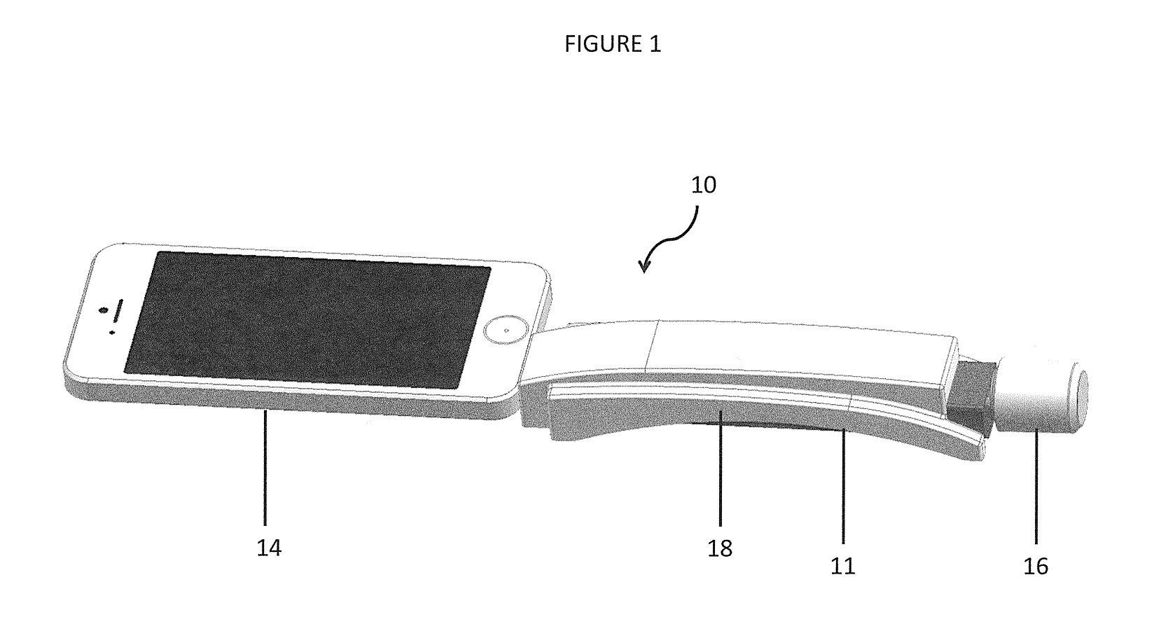

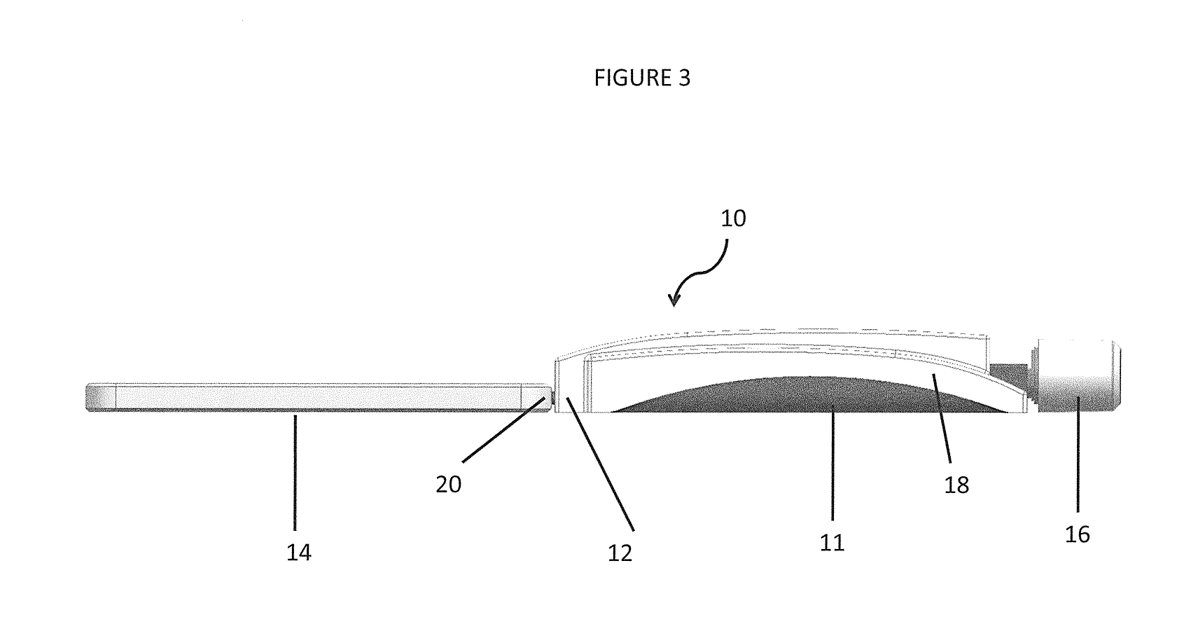

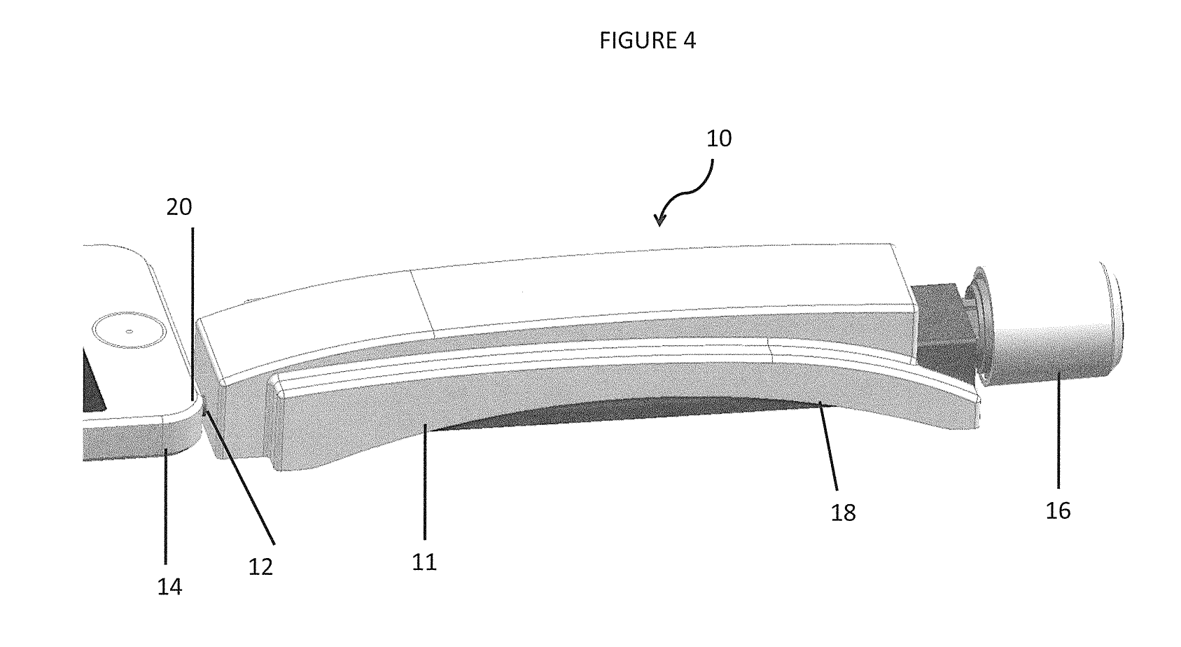

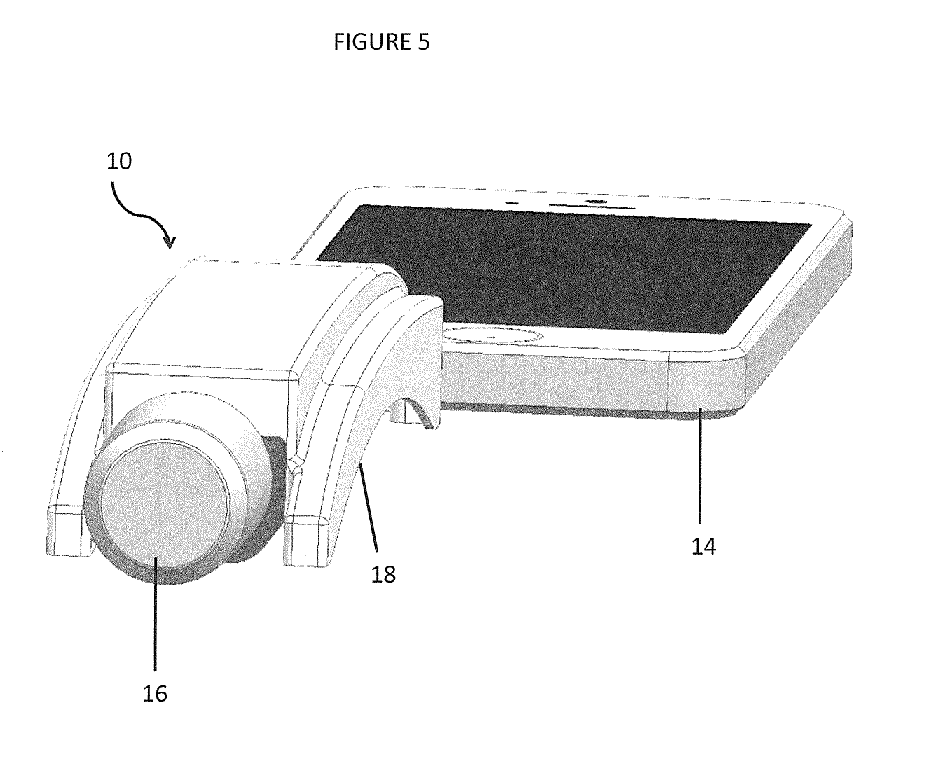

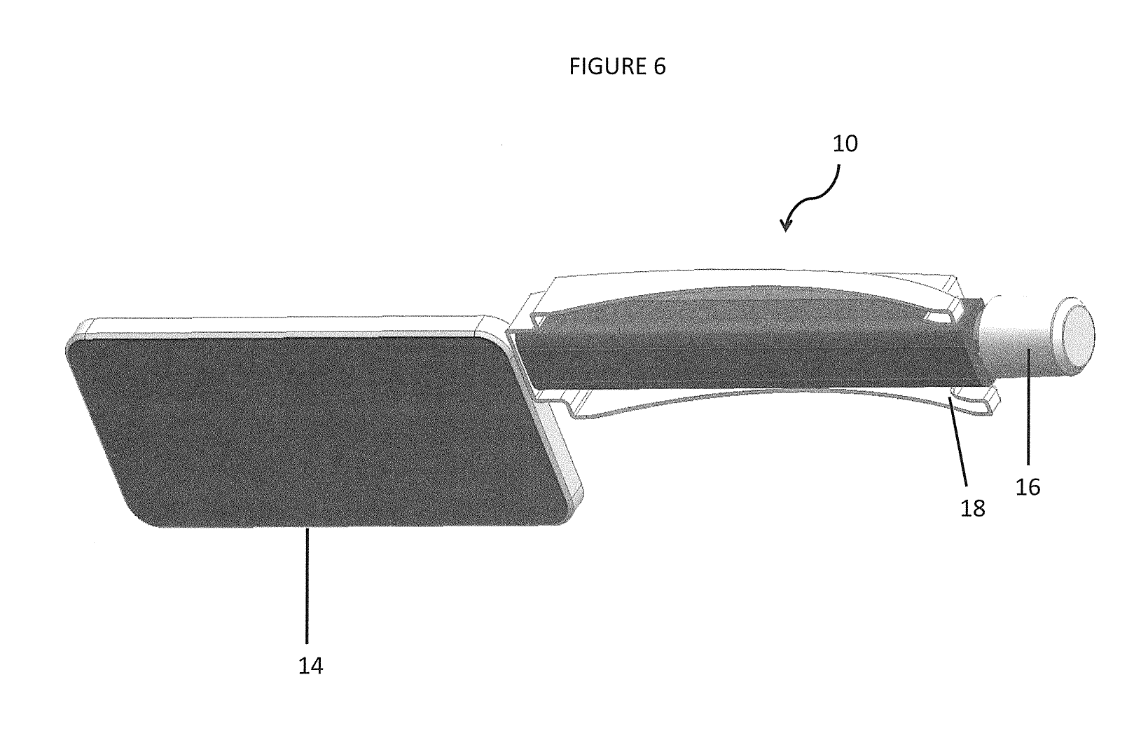

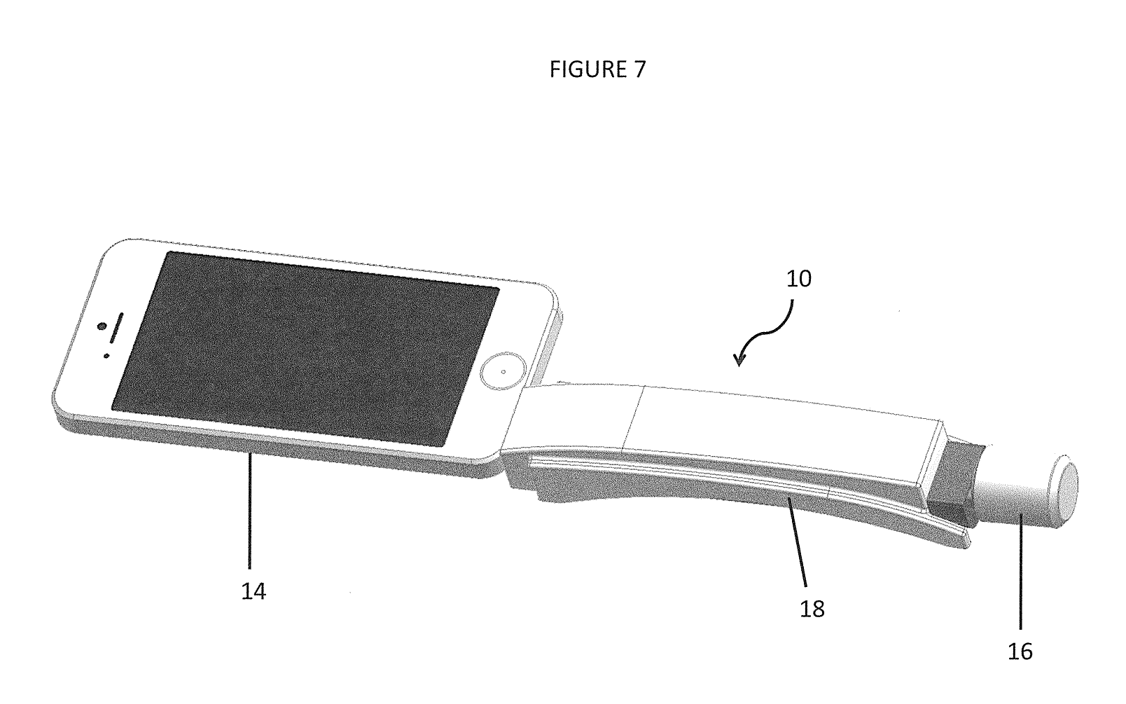

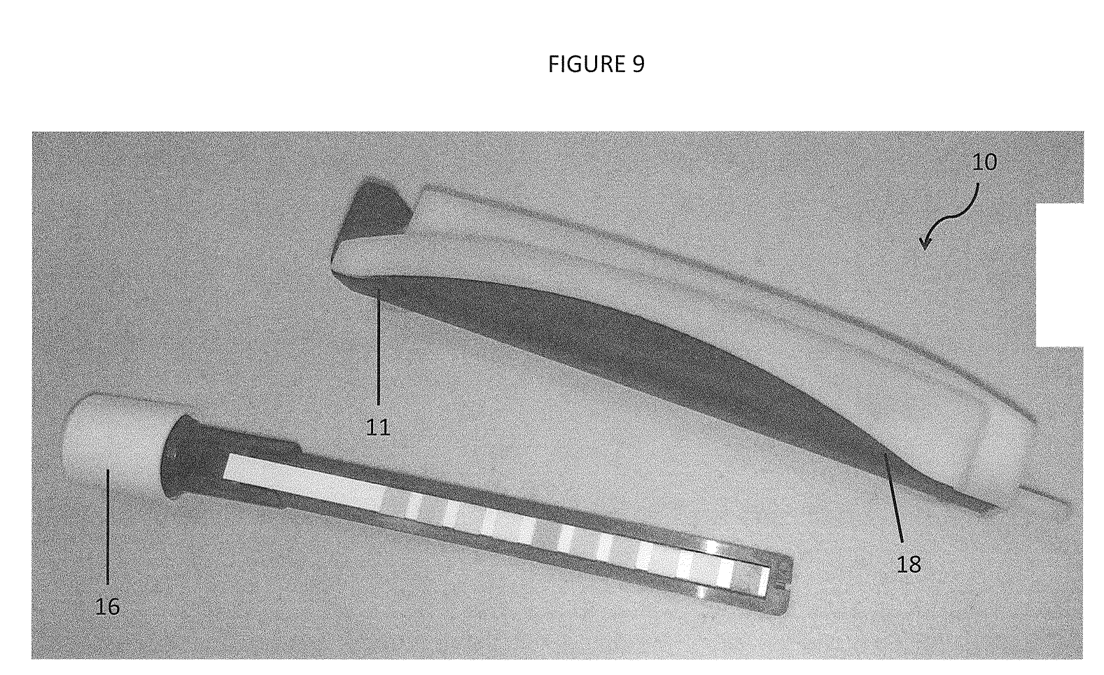

The present invention generally provides for a urinalysis device, shown at 10 in FIGS. 1-7 and 9-10, that includes a device body 11 having a port 12 for electronic communication with a handheld computing device 14.

“App” or “Application” as used herein, refers to a software program designed to run on the operating systems of a handheld or mobile computing device.

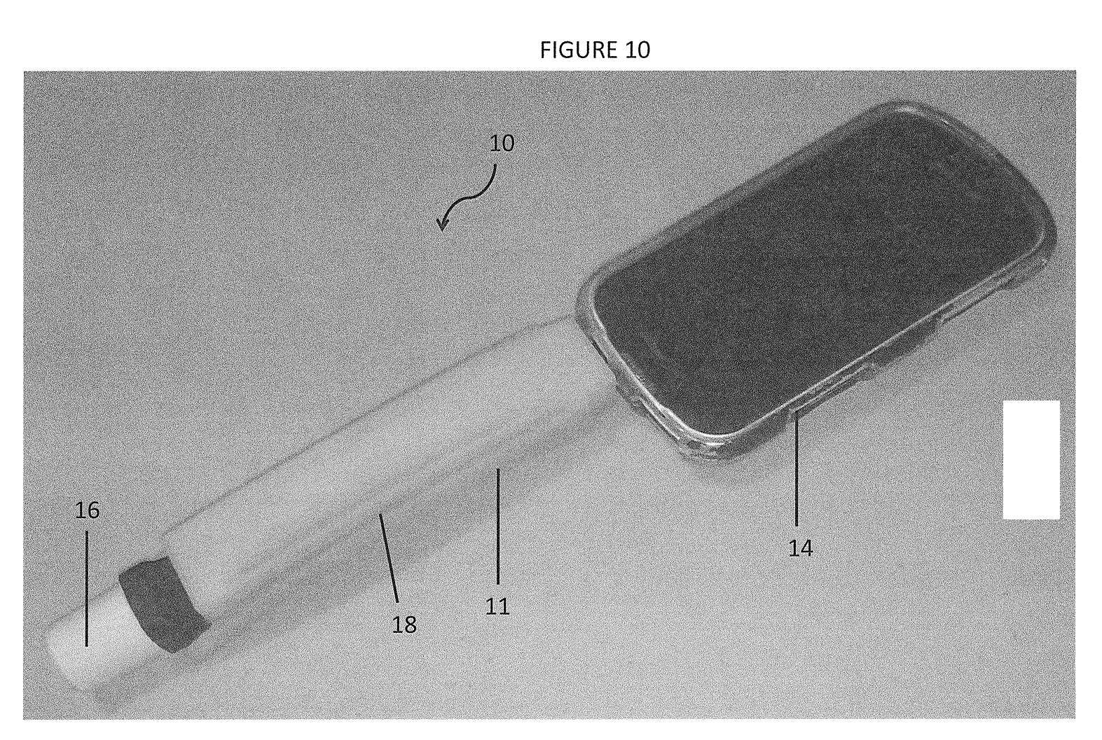

The urinalysis device 10 can be any suitable handheld urinalysis device or wellness panel including a test strip 20 (shown in FIG. 10 housed in holder 16) that is inserted into a stream of urine in order to take a sample for various tests. Preferably, the urinalysis device 10 includes a mechanism that can read the test strip 20, such as a plurality of LEDs and photo-sensors (preferably 10 LEDs and 10 photo-sensors) (not shown) that can read the test strip in the device body 11. Preferably, the device body 11 is a plastic carrier 18. The urinalysis device can alternatively read a DIASCREEN® 10 (Arkray, standard urinalysis strip) placed into the plastic carrier 18. The plastic carrier 18 can have any suitable combination of light sources and sensors or sensor array in order read the test strip 20. The plastic carrier 18 is shaped in any suitable configuration to securely receive the holder 16, also preferably made of plastic.

The test strip 20 can include one or more individual test pad sites to be analyzed by the urinalysis device 10, and include (at least one) tests such as, but not limited to, inflammation monitoring tests, oxidative stress monitoring tests, and antioxidant activity monitoring tests for humans and/or animals, and combinations thereof. Biomarkers quantified by these tests can include, but are not limited to, TNF-α, IL-6, IL-8, osteopontin, orosomucoid, albumin, α1-microglobulin, PGE₂, PGF_{2α}, nitric oxide, nitrate and nitrate derived from nitric oxide (NOx), histamine, urinary protein, protein carbonyls, thiobarbituric acid reactive substances (TBARS), malonaldehyde, 4-hydroxynonenal, lipid hydroperoxides, isoprostanes, linoleic acid oxidation products, nitrotyrosine, nitrothiols, 8-hydroxy-deoxyguanosine, M1dG, oxidized derivatives of the ribose ring, selenium, GSH, GSSG, the GSH/GSSG ratio, uric acid, glutathione peroxidase, superoxide dismutase, ascorbic acid, and combinations thereof. The test strip 20 can be made of materials known in the art, such as, but not limited to, a test strip of hydrophobic backing material with blotting capabilities to blot excess fluid for minimal crosstalk.

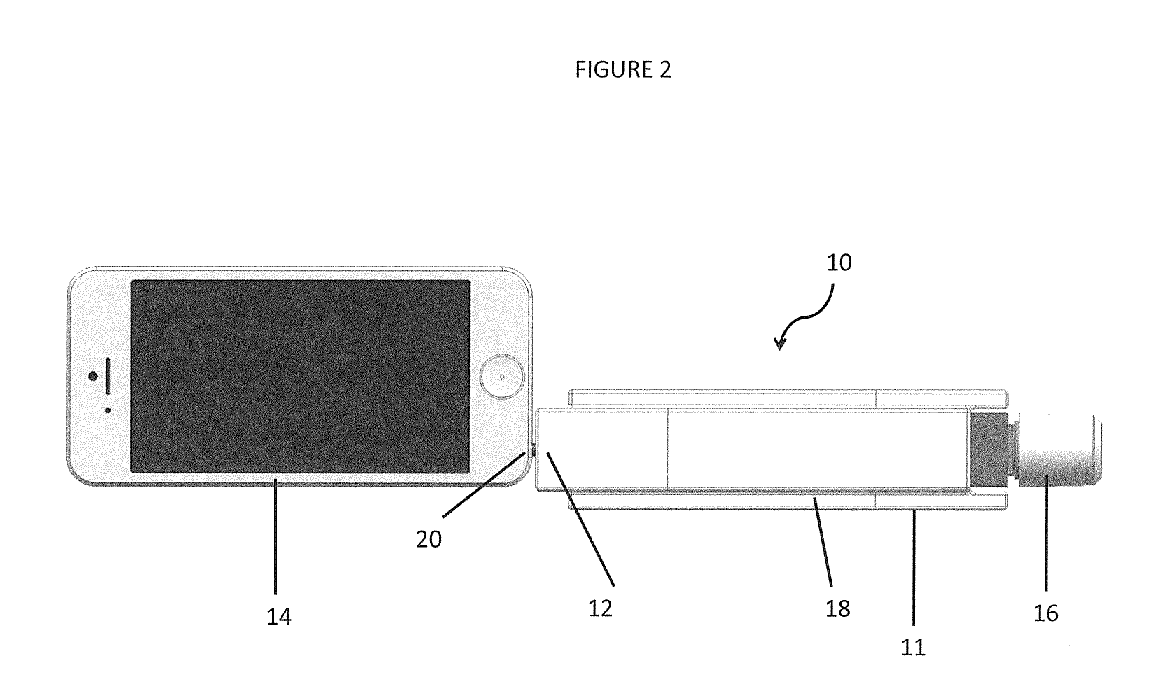

The port 12 can be any suitable electronic port that allows coupling to the handheld computing device 14 at any suitable site on the handheld computing device 14, best seen in FIG. 2. For example, the port 12 can include necessary hardware to electronically and physically couple to a mic/headphone output 20 on the handheld computing device 14. The port 12 can alternatively include necessary hardware to electronically and physically couple to a data/charging port (not shown) on the handheld computing device 14. Various connectors or hardware interfaces can be included for coupling and providing power and data transfer, such as, but not limited to, usb connectors (including standard usb and usb-c), micro usb connectors, 30-pin connectors, FIREWIRE® (Apple) (IEEE-1394) connectors, THUNDERBOLT® (Intel) connectors (i.e. connectors that combine PCI Express and DisplayPort into one serial signal and provide DC power), LIGHTNING® (Apple) (8-pin) connectors, and combinations thereof. It should be understood that the various mic/headphone outputs 20 and data/charging port can be located in different places on different handheld computing devices 14.

The handheld computing device 14 can be any suitable smartphone (such as, but not limited to, an (PHONE® (Apple)), tablet (such as, but not limited to, an (PAD® (Apple), or Microsoft SURFACE® tablet), or handheld or mobile device that allows for the use of Apps (such as, but not limited to, portable mp3 players and smart watches) and provides a graphical interface with which to display readings from the urinalysis device 10.

In use, a urine sample is taken with the urinalysis device 10 (and if necessary, any test strip 16 or holder 22 is inserted into the plastic carrier 18). The urinalysis device 10 is electronically coupled to the handheld computing device 14 through the port 12, raw data is obtained from the test strip 16 within the urinalysis device 10, and the raw data is sent to the handheld computing device 14 and further analyzed, preferably with an Application on the handheld computing device 14.

Once electronically coupled, all power for the urinalysis device 10 is drawn from the handheld computing device 14 through port 12 at either the mic/headphone output 20 or the data/charging port. Power thus derived drives and operates the LEDs and photo-sensors in the urinalysis device 10, which transmits raw reflectance or RGBW values to a tandem App in order to analyze the urine sample on the test strip 16. Many different substances within the urine can be analyzed with any suitable test. The urinalysis device 10 itself can have any required internal processing, power management, or data output capabilities, or alternatively it can rely on a tandem App installed on the urinalysis device 10 to do so.

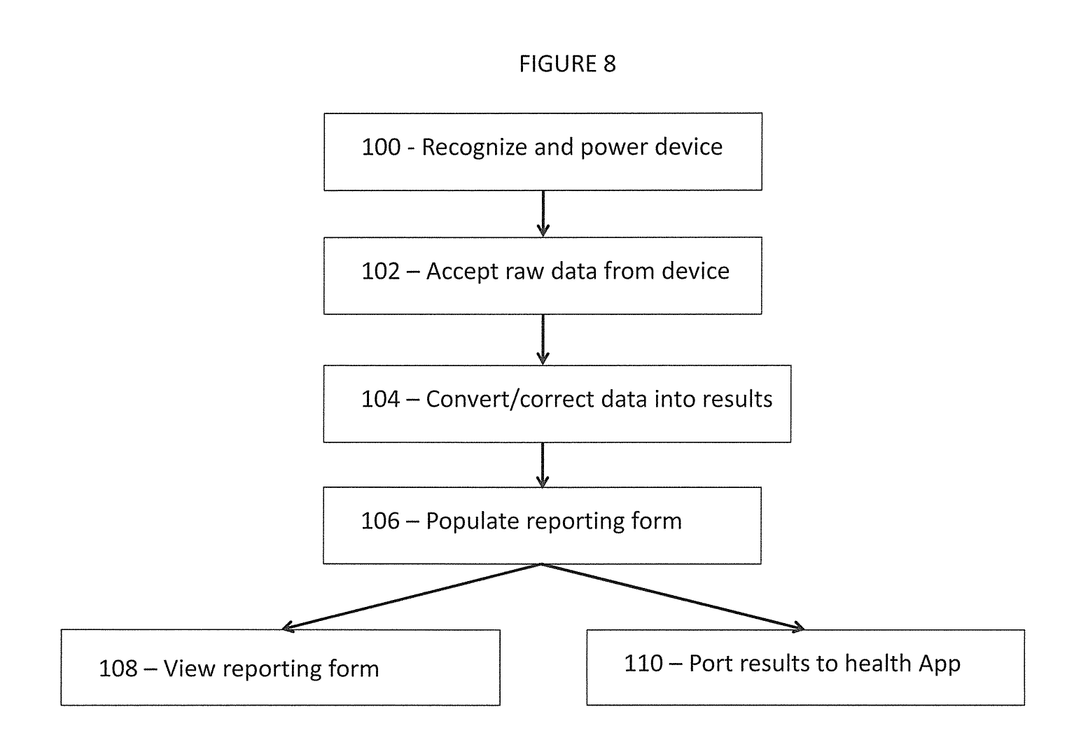

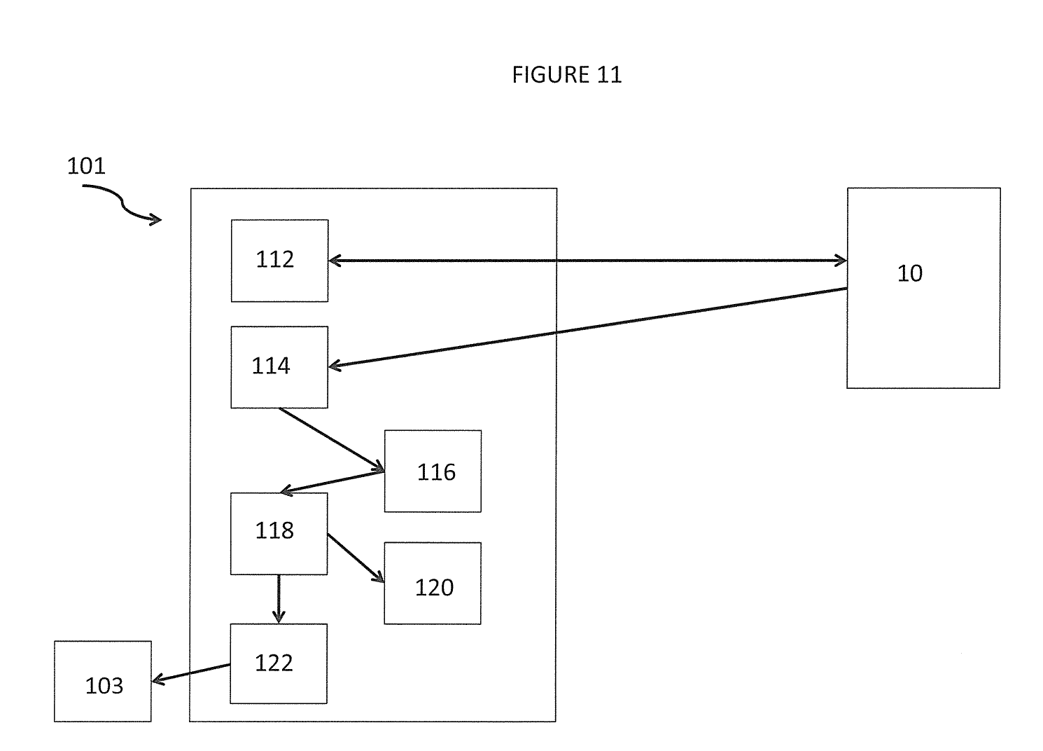

The urinalysis device 10 operates in tandem with an App 101 stored on computer readable media (i.e. any non-transitory computer readable media or hardware such as flash memory drives, RAM, ROM, etc.) within the handheld computing device 14 with specific features, capabilities, and instructions, including, but not limited to, the following, and shown in FIG. 8 and FIG. 11. The method of use of the App and the physical components of the App are as follows. The App recognizes and provides power (100) to the urinalysis device 10 when the port 12 has been electronically coupled to the handheld computing device 14 with a power providing mechanism 112. The App accepts raw data (102) from the urinalysis device 10 that has been obtained from the LEDs and photo sensors with a data accepting mechanism 114 and subsequently converts and/or corrects this raw data (104) from the urinalysis device 10 to useful results of information and data with a data converting mechanism 116. The results/data are then populated in a reporting form (106) resident in the App itself with a reporting mechanism 118. Therefore, the results of the urine sample can be read/viewed (108) if desired directly on a display in the App with a displaying mechanism 120.

However, the App can also port any results (110) directly into an outside location 103 such as a separate health App (such as, but not limited to, Apple's “HealthTrackr®” App with permission, using Apple's developer kit to create the ports and permissions), optionally, and port results to any other suitable health monitoring Apps capable of accepting data, or any other medical records database, devices, networks, or websites in an encrypted file with a porting mechanism 122. Therefore, the user of the App can obtain urinalysis results and view them in context of any other health data collected by other Apps or sources. The App can thus be in electronic communication with a separate health App, medical records database, devices, networks, or websites.

Alerts (sound, light) can be created within any App to notify the user that results are ready, or any values are determined to be high or unhealthy (based on a baseline). Results can also be sent to computing platforms of healthcare professionals via the Internet or wireless signals from the App.

Most preferably, the App is made available from the Apple App Store and is downloaded to the handheld computing device 14 for free by entering in a serial number of the urinalysis device 10 plus a key number (in order to prevent people from sharing the urinalysis device 10).

The urinalysis device 10 can be instantly re-programmed and re-purposed by adding other software applications which are designed to use the same platform, i.e. a multi-pad urine strip in a cartridge form and the urinalysis device 10, to conduct other urinalysis tests, namely a standard urinalysis strip (referred to as a “10-way”) that includes, but is not limited to, leukocytes, nitrogen, uroalbumin, protein, pH, blood, specific gravity, ketones, bilirubin, and glucose for human testing and other biomarkers.

The urinalysis device 10 can also test humans for fat metabolization, muscle efficiency, glycemic dysregulation, and metabolic efficiency with a multi-site/multi-reagent test strip 16 developed specifically for the urinalysis device 10 for athletic and military training purposes that can be ported to a variety of other smart devices, including tablets, laptops, smart watches, wearable computers, etc.

The urinalysis device 10 can be used as a peripheral for a tele-medicine system. The urinalysis device 10 allows old or currently existing devices that merely measure sickness (i.e. blood pressure cuff, heart rate monitor, EKG, etc.) to now determine wellness.

The urinalysis device 10 is advantageous because it provides a quick and easy way to obtain results of a urine test (within a matter of minutes in any setting) and the App makes it easy to view results and collect results with other health measurements. The non-invasive tests on the urinalysis device 10 can be performed at a fraction of the cost of lab-based tests and the results can easily reach the patient and health care professionals through the use of the App.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

1.-27. (canceled)

28. A urinalysis device comprising:
a device body configured for holding an assembly of a urinalysis test strip and a test strip holder, the device body comprising a port for physical and electronic coupling with a handheld computing device;
an assembly held in the device body and comprising a urinalysis test strip and a test strip holder, the urinalysis test strip configured for insertion into urine and collecting a urine sample; and
at least one test pad site on the urinalysis test strip,
wherein the at least one test pad site includes at least one test selected from the group consisting of inflammation monitoring tests, oxidative stress monitoring tests, antioxidant activity monitoring tests, and combinations thereof.

29. The urinalysis device of claim 28, wherein the at least one test pad site includes at least one test that quantifies a biomarker chosen from the group consisting of TNF-α, IL-6, IL-8, osteopontin, orosomucoid, albumin, α1-microglobulin, PGE₂, PGF_{2α}, nitric oxide, nitrate and nitrate derived from nitric oxide (NOx), histamine, urinary protein, protein carbonyls, thiobarbituric acid reactive substances (TBARS), malondialdehyde, 4-hydroxynonenal, lipid hydroperoxides, isoprostanes, linoleic acid oxidation products, nitrotyrosine, nitrothiols, 8-hydroxy-deoxyguanosine, M1dG, oxidized derivatives of the ribose ring, selenium, GSH, GSSG, the GSH/GSSG ratio, uric acid, glutathione peroxidase, superoxide dismutase, ascorbic acid, and combinations thereof.

30. The urinalysis device of claim 28, wherein the device body further comprises a plurality of LEDs and photo-sensors in optical alignment for reading the urinalysis test strip.

31. The urinalysis device of claim 28, wherein the device body comprises a plastic carrier.

32. The urinalysis device of claim 28, wherein the port comprises hardware for electronically and physically coupling the urinalysis device to the handheld computing device at a site on the handheld computing device selected from the group consisting of a mic/headphone output and a data/charging port.

33. The urinalysis device of claim 32, wherein the hardware is selected from the group consisting of usb connectors, micro usb connectors, 30-pin connectors, IEEE-1394 connectors, connectors that combine PCI Express and DisplayPort into one serial signal and provide DC power, 8-pin connectors, and combinations thereof.

34. The urinalysis device of claim 28, further comprising a handheld computing device coupled to the port, the handheld computing device selected from the group consisting of a smartphone, a tablet, a portable mp3 player, and a smart watch.

35. The urinalysis device of claim 28, wherein the urinalysis test strip includes tests for leukocytes, nitrogen, uroalbumin, protein, pH, blood, specific gravity, ketones, bilirubin, and glucose.

36. A method of analyzing a urine sample using a handheld computing device and a urinalysis device configured for coupling thereto, said method comprising the steps of:
inserting a urinalysis test strip into the urine sample and placing the urinalysis test strip into the urinalysis device, the urinalysis device comprising a device body configured for holding the urinalysis test strip, the device body comprising a port for physical and electronic coupling with the handheld computing device;
electronically coupling the urinalysis device to the handheld computing device through the port;
obtaining raw data from the urinalysis test strip within the urinalysis device;
sending the raw data to the handheld computing device; and
analyzing the raw data by the handheld computing device.

37. The method of claim 36, wherein the step of obtaining raw data from the urinalysis test strip further comprises operation of LEDs and aligned photo-sensors in the urinalysis device powered by the handheld computing device.

38. The method of claim 36, wherein the step of sending the raw data to the handheld computing device further comprises transmitting at least one of raw reflectance and RGBW values to an Application residing on non-transitory computer readable media of the handheld computing device.

39. The method of claim 36, wherein the urinalysis test strip comprises at least one test pad site further comprising at least one test chosen from the group consisting of inflammation monitoring tests, oxidative stress monitoring tests, antioxidant activity monitoring tests, and combinations thereof.

40. The method of claim 36, wherein the urinalysis test strip comprises at least one test that quantifies a biomarker chosen from the group consisting of TNF-α, IL-6, IL-8, osteopontin, orosomucoid, albumin, α1-microglobulin, PGE₂, PGF_{2α}, nitric oxide, nitrate and nitrate derived from nitric oxide (NOx), histamine, urinary protein, protein carbonyls, thioberbituric acid reactive substances (TBARS), malondialdehyde, 4-hydroxynonenal, lipid hydroperoxides, isoprostanes, linoleic acid oxidation products, nitrotyrosine, nitrothiols, 8-hydroxy-deoxyguanosine, M1dG, oxidized derivatives of the ribose ring, selenium, GSH, GSSG, the GSH/GSSG ratio, uric acid, glutathione peroxidase, superoxide dismutase, ascorbic acid, and combinations thereof.

41. The method of claim 36, wherein the Application comprises: a power providing mechanism for recognizing and providing power to the urinalysis device; a data accepting mechanism for accepting raw data from the urinalysis device; a data correcting mechanism for converting and correcting raw data from the urinalysis device to results; and a reporting mechanism for populating a reporting form resident in the Application with results.

42. The method of claim 36, wherein the Application further comprises a displaying mechanism for displaying results in the application.

43. The method of claim 36, wherein the Application further comprises a porting mechanism for porting the results directly into an outside location selected from the group consisting of a health Application, a medical records database, a device, a network, and a website.

44. A method of using a urinalysis device with an Application on non-transitory computer readable media, said method comprising the steps of:
recognizing and providing power to the urinalysis device;
accepting raw data from the urinalysis device;
converting and correcting raw data from the urinalysis device to results; and
populating a reporting form resident in the Application with results.

45. The method of claim 44, further comprising the step of displaying results in the Application.

46. The method of claim 44, further comprising the step of porting the results directly into an outside location chosen from the group consisting of a health Application, a medical records database, a device, a network, and a website.

47. The method of claim 44, further comprising the step of providing an alert regarding results.

48. A system comprising:
(1) a handheld computing device further comprising a computer readable media;
(2) a urinalysis device coupled with the handheld computing device via connection between a port on the urinalysis device and a site on the handheld computing device, the urinalysis device comprising:
(a) a device body configured for holding an assembly comprising a urinalysis test strip and a test strip bolder,
(b) an assembly held in the device body and comprising a urinalysis test strip and a test strip holder, the urinalysis test strip configured for insertion into urine and collecting a urine sample; and
(c) at least one test pad site on the urinalysis test strip; and
(3) a computer application stored on the computer readable media, the computer application comprising a data accepting mechanism for accepting raw data transmitted from the urinalysis device to the handheld computing device; and a reporting mechanism for populating a reporting form resident in the application with results.

49. The system of claim 48, wherein the device body further comprises a plurality of LEDs and photo-sensors in optical alignment for reading the urinalysis test strip and generating the raw data.

50. The system of claim 48, wherein the at least one test pad site includes at least one test selected from the group consisting of inflammation monitoring tests, oxidative stress monitoring tests, antioxidant activity monitoring tests, and combinations thereof.

51. The system of claim 48, wherein the at least one test pad site includes at least one test that quantifies a biomarker chosen from the group consisting of TNF-α, IL-6, IL-8, osteopontin, orosomucoid, albumin, α1-microglobulin, PGE₂, PGF_{2α}, nitric oxide, nitrate and nitrate derived from nitric oxide (NOx), histamine, urinary protein, protein carbonyls, thiobarbituric acid reactive substances (TBARS), malondialdehyde, 4-hydroxynonenal, lipid hydroperoxides, isoprostanes, linoleic acid oxidation products, nitrotyrosine, nitrothiols, 8-hydroxy-deoxyguanosine, M1dG, oxidized derivatives of the ribose ring, selenium, GSH, GSSG, the GSH/GSSG ratio, uric acid, glutathione peroxidase, superoxide dismutase, ascorbic acid, and combinations thereof.

52. The system of claim 48 wherein the system is a tele-medicine system.

53. The system of claim 48 wherein the system is a telehealth system.
